(19) United States
(12) Patent Application Publication
(10) Pub. No.: US 2012/0157862 A1
(43) Pub. Date: Jun. 21, 2012

(54) PRESSURE TRANSDUCER FOR MEDICAL USE AND CONTACT HOLDER

(75) Inventors: Bernd Beck, Rangendingen (DE); Alojz Simoncic, Sentjernej (SI)

(73) Assignees: HYB D.O.O., Sentjernej (SI); Bernd Beck, Rangendingen (DE)

(21) Appl. No.: 13/393,054

(22) PCT Filed: Aug. 12, 2010
(86) PCT No.: PCT/EP10/61768

§ 371 Date: Feb. 28, 2012

(30) Foreign Application Priority Data
Aug. 28, 2009 (DE) 10 2009 039 958.5

(51) Int. Cl. A61B 5/0215

(52) U.S. Cl. 600/488

(57) ABSTRACT

The invention relates to a pressure transducer for measuring blood pressure for medical use, which can be connected by pressure to the vascular system of a patient, said transducer comprising a pressure measuring element for measuring the prevailing pressure in the fluid chamber, and also mechanical holding means for detachably mounting the pressure transducer onto the contact holder. The invention also relates to a contact holder for holding and for electrically contacting a pressure transducer for medical use.

### BACKGROUND OF THE INVENTION

The invention relates to a pressure transducer for measuring blood pressure for medical use and to a contact holder for mounting and electrically contacting pressure transducers for medical use.

Such pressure transducers usually have a fluid chamber that can be connected by pressure to the vascular system of a patient. Here, fluid tubes which are filled with a physiological electrolyte solution (e.g. NaCl solution) can be used for the connection by pressure. In the process, the fluid chamber is connected to a pressure measuring element for converting the pressure into electrical measurement signals. Accordingly, in order to operate such a pressure transducer it is necessary to contact the device electrically and, at the same time, connect it to a system filled with liquid.

DE 198 51 274 A1 discloses a pressure transducer with an electrical plug-in connection. Here, a pin-like plug can be inserted into a socket, with provision being made on the socket opening for an elastic sealing element that is pre-punctured in the insertion direction. The object of the sealing element is, on the one hand, to seal the opening of the socket in a substantially liquid-tight fashion and, on the other hand, to wipe possible liquid drops off the plug when said plug is inserted.

Particular demands are placed on pressure transducer arrangements in medical use. Relatively long examinations or treatments of different patients require frequent replacements of the pressure transducers, particularly for reasons of hygiene. In this process, there can easily be confusing situations as a result of the multiplicity of fluid tubes and cables for electrically contacting the pressure transducers, which can lead to inadvertent severing of connections and, not least, a risk to the health of the patient. As a result of the frequent changes, the pressure transducers are consumables, which should preferably therefore, as mass-produced articles, be producible in a cost-effective fashion. Complicated mechanical structures with many components, e.g. delicate sealing elements on plug sockets, are therefore as a matter of principle undesirable for the consumable pressure transducer, particularly for reasons of cost.

When the pressure transducers are replaced, the pressure transducer arrangement can easily be contaminated by liquid from the fluid system. Since the utilized physiological electrolyte solutions have good electric conductivity, this creates the risk of short circuits and/or shunts, particularly in the region where the pressure transducer is contacted electrically. This can lead to changes in the measured current, voltage and resistance values and hence to a falsification of the measured values. Moreover, permanent contact with electrolyte solution can lead to corrosion of metallic contact faces and other electric components. In respect of this problem, complete sealing of the plug socket or keeping the plug completely dry is, in principle, difficult or cannot be achieved using plug-in connections with sealing elements as known from the prior art.

### SUMMARY OF THE INVENTION

The invention is based on the object of providing a pressure transducer arrangement which allows mounting and electrical contacting of pressure transducers in a simple and robust fashion and allows fault-free operation, even under the influence of liquids.

Accordingly, the pressure transducer according to the invention is distinguished by the fact that provision is made for an electrically insulating base plate into which at least one electric contact face or contact that is electrically connected to the pressure measuring element has been embedded such that the surfaces of the contact face and the base plate substantially lie in a common plane and that an electrical contact is established between the contact face and an associated counter contact of the contact holder by mounting the pressure transducer in the contact holder.

Moreover, the pressure transducer can be mechanically mounted in a detachable fashion, wherein, when mounted, the pressure transducer is also contacted electrically via the contact faces embedded into the base plate. It follows that there is no need for additional cables and/or plug-in connections for contacting the pressure transducer, which results in the assembled pressure transducer arrangement having a comparatively clear embodiment and thus increases the treatment safety. By way of example, it is feasible that the pressure transducer is inserted or clamped into a correspondingly designed contact holder, with the base plate being brought to adjoin a support section of the contact holder such that the contact faces are contacted.

Moreover, the pressure transducer according to the invention has a comparatively simple and robust design. By way of example, the base plate with embedded contact faces, the holding means, the pressure measuring element and the fluid chamber can be arranged above one another in a layer-like fashion. This makes the pressure transducer incredibly compact. Complex geometric embodiments, such as e.g. plug sockets or plug pins, can be dispensed with. Furthermore, components can be saved compared to known pressure transducers; thus, for example, if the contact faces are embedded into the insulating base plate there is no need for additional sealing or insulating elements. Thus, overall, the pressure transducer according to the invention is suitable for a cost-effective production process that is suitable for mass production.

Since the surfaces of the contact face and the base plate substantially lie in a common plane, they adjoin one another without steps. The whole surface or the base plate as a whole can in this case also have a curved design. In any case, this avoids gaps or recesses in which liquid or contaminants could collect—which can, for example in the case of plug/socket contacts, lead to the above-described faulty operation and to corrosion. The contact faces on the base plate of the pressure transducer according to the invention are freely accessible and can therefore be cleaned quickly and easily, and can be freed from possible liquid accumulations.

Here the base plate is preferably formed from a ceramic material that has the required hardness and stiffness, is produced cost-effectively and acts in an electrically insulating fashion. Moreover, it is easy to embed metallic contact faces into the ceramics, for example by molding, when the base plate is produced. Moreover, electrical circuit elements, e.g. functional elements for the pressure measuring element, can be applied onto ceramic materials using known technical methods (e.g. lithography).

The contact face is advantageously embedded into the base plate in a liquid-tight fashion. By way of example, embedding the contact face in an integral fashion during the production process is feasible. The contact face can also be worked into the base plate in a cohesively bonded fashion so that no additional measures are required for sealing. The unit comprising base plate and embedded contact face thus has a substantially smooth surface, which can be easily cleaned and is robust.

For the further embodiment, provision can be made for an elastic pressure pad which is arranged for transmitting the pressure between the fluid chamber and the pressure measuring element. The pressure pad can moreover be formed from a liquid-tight material such that the electronics of the pressure measuring element is sealed with respect to the fluid chamber and the functional reliability can be increased thereby.

A particularly preferred embodiment provides for the base plate to have electric circuits of the pressure measuring element. More particularly, the base plate has all essential or all functional parts of the measurement electronics, or it comprises the pressure measuring element. By way of example, the electric circuits can in this case be applied to the base plate using lithographic methods. This results in a particularly compact design of the pressure transducer and a cost-effective production method suitable for mass production.

It is particularly preferred for the fluid chamber and the pressure measuring element to be arranged together on one side of the base plate and for the at least one contact face to be accessible from the side of the base plate distant from the fluid chamber. A pressure transducer embodied thus can be inserted into a contact holder for being contacted electrically, with the functional parts (fluid chamber, pressure measuring element) facing away from the contact holder and thus being accessible for possible required handling.

Advantageously, provision is made for a fluid tube for connecting the pressure transducer to the patient and the fluid chamber is provided by a section of the fluid tube. This results in a further simplification of the design since no additional measures are required for connecting the fluid chamber to the fluid tube and/or for sealing this connection. The fluid tube can directly adjoin the pressure measuring element, or interact with the pressure measuring element via a pressure pad or further intermediate elements.

For the further embodiment, provision is made for the base plate to provide a holding section, which provides the holding means for the pressure transducer. In this case, holding edges or latching lugs on the base-plate side are feasible. Moreover, it is also feasible for the pressure transducer to have a housing or base part and a holding section providing the holding means, which holding section is arranged on the housing or base part. This also contributes to the further simplification of the design.

The object posed at the outset is also achieved by a contact holder for mounting and electrically contacting a pressure transducer for medical use. The contact holder provides for a plate-like support section, which has at least one contact pin, which has a contact tip as electrical counter contact for a contact face of the pressure transducer, wherein the contact pin is arranged substantially perpendicular to the support section such that the contact tip is distant from the support section and contacts the associated contact face if the pressure transducer is mounted. Here, moreover, provision is made for a sealing membrane made of an elastic, hydrophobic and electrically insulating material, which sealing membrane is arranged adjoining the support section and has a recess for respectively one contact pin such that the contact pin is embedded into the sealing membrane in a liquid-tight fashion and the contact tip remains uncovered by the sealing membrane.

The sealing membrane is preferably made of a polysiloxane (silicone) which is elastic, electrically insulating and repellent to polar liquids (hydrophobic) and moreover can be produced in a cost-effective fashion in almost any shape. However, other elastomeric materials are also feasible. In particular, the sealing recesses for the contact pins can be easily implemented when producing the sealing membrane from a polysiloxane (silicone).

By mounting a pressure transducer in a contact holder according to the invention, an electric connection can be obtained without additional cables or plug-in connections, which has the advantages as already explained above in respect of the pressure transducer according to the invention.

The contact holder also has a comparatively simple and compact design because only substantially planar components are assembled adjoining one another. A cost-effective production is possible once again, even as a mass-produced article.

Since the contact pins are embedded into the recesses in a liquid-tight fashion and it is only the contact tips that remain uncovered, the contact region of the contact holder has a substantially continuous, smooth surface. The accumulation of liquids and contaminants in gaps or recesses is largely avoided. Moreover, it is easy to clean the electrical contact region.

As explained at the outset, contaminations with the liquid of the fluid system for measuring pressure in particular constitute a problem for electrical contacting. For the fluid, use is usually made of physiological electrolytes (e.g. NaCl solution), which are usually polar and electrically conductive, aqueous solutions. Since the sealing membrane has a hydrophobic embodiment, large-area wetting of the contact region by the aforementioned fluids is largely avoided. On the one hand, this simplifies cleaning in the case of contamination or wetting, and it can prevent corrosion resulting from constant contact with liquid. On the other hand, the hydrophobic and electrically insulating sealing membrane reduces the risk of short circuits or shunts between adjacent contact pins and/or contact faces, without this requiring further components for sealing or insulation (as occurs, for example, in the form of a sealing element in the case of the known plug/socket contacts).

A pressure transducer according to the invention is preferably mounted in the contact holder such that the base plate and contact faces are pressed against the sealing membrane and contact tips, respectively. Since the sealing membrane has an elastic embodiment, possibly present drops of liquid are in the process pressed out of the contact region. This largely avoids the risk of short circuits or shunts, particularly in conjunction with the hydrophobic embodiment of the sealing membrane, and so fault-free operation is made possible, even in moist surroundings.

A particularly preferred embodiment of the contact holder emerges from the fact that, around the recess, the sealing membrane has an annular sealing bead that adjoins the contact pin in a sealing fashion. In particular, it is advantageous if the sealing bead is dimensioned such that it ends flush with the contact tip such that the contact tip and the sealing bead rise to the same extent above the surface of the sealing membrane.

When the pressure transducer is mounted, the sealing bead displaces possibly present liquid in the region around the contact pin. This can largely prevent the risk of inadvertent short circuits or shunts to other contact pins and/or contact faces.

Provision is preferably made for the contact pin to comprise a cylindrical shaft and a pin head with a greater radial diameter than the shaft, wherein the pin head has the contact tip and adjoins the sealing membrane and wherein the sealing membrane encloses the shaft in a liquid-tight fashion. The recess that surrounds the shaft of the contact pin in a liquid-tight fashion is additionally covered by the pin head with the greater diameter. As a result, the sealing effect of the sealing membrane against the ingress of liquid into the region between various contact pins is increased.

The contact tip advantageously has a mushroom-like design and/or is rounded-off like a cap. This avoids points and/or sharp edges, which could for example lead to damage to contact faces of the pressure transducer or to the fluid tubes.

A particularly preferred development of the invention emerges from the contact pin being provided on the support section such that the contact tip can be depressed in the direction of the support section against tensioning applied by a spring element. By way of example, the spring element can in this case be provided by the elastic sealing membrane itself, the surface of which is adjoined by the contact tip more particularly embodied as pin head with a relatively large radial diameter. It is also feasible for a spring element to be arranged on or around the contact pin, which acts on the contact tip counter to the support part. If a pressure transducer is mounted in such a contact holder, the arrangement of the contact tip that can be depressed under tensioning can ensure that the contact tips are pressed against the corresponding contact faces when a pressure transducer is inserted. This provides good electrical contact, even if the contact faces and/or the contact tips are dirty or damp. This increases the functional reliability of the pressure transducer arrangement.

For the further embodiment of the contact holder, provision is made for a holding device such that a pressure transducer can be inserted parallel to the support section for the purpose of being detachably mounted on the contact holder. When using the contact holder with a pressure transducer according to the invention, provision is made, in particular, for the pressure transducer to be able to be inserted into the holding device with the base plate of the former being parallel to the support section. This can achieve quick, simple and re-detachable mounting of the pressure transducer. In particular, it is advantageous if the base plate of the pressure transducer is pressed against the sealing membrane of the contact holder during the insertion. This wipes liquid drops off the contact region and thus avoids malfunctions as a result of short circuits or shunts.

The contact holder advantageously comprises an electric printed circuit board, which is arranged adjoining the support section or parallel to the support section on the side distant from the sealing membrane such that the contact pin is electrically contacted. Here, the printed circuit board may contain electric printed conductors, by means of which the electric signals from the contact pins are transmitted to a signal-routing device, e.g., a bundle of cables. It is also feasible that the printed circuit board has electric circuits for processing, filtering or amplifying the signals. By way of example, the printed circuit board can be screwed or adhesively bonded to the support section on the side of the latter distant from the sealing membrane such that the contact pins are correspondingly contacted. This results in a simple and compact, layer-like design for the contact holder.

It is particularly preferred for the contact pin to have an annular groove and, in the region of the recess, the sealing membrane to have an annular projection, which engages into the annular groove. In particular, the annular projection engages into the annular groove such that the contact pin is mounted in the sealing membrane in an axially moveable fashion. The annular projection advantageously has a design that is matched to the annular groove. As a result, the engagement of the annular projection into the annular groove can ensure that the contact pin is embedded into the sealing membrane in a liquid-tight fashion.

The advantages illustrated above are particularly pronounced in a pressure transducer arrangement that comprises a pressure transducer according to the invention and a contact holder according to the invention. Here, the contact faces in the base plate of the pressure transducer are arranged matched to the contact pins embedded in the sealing membrane of the contact holder. Here, the contact holder can for example be integrated into the housing of a data evaluation and transmission unit of a patient monitoring system.

### BRIEF DESCRIPTION OF THE DRAWINGS

Further details and advantageous embodiments of the invention can be gathered from the following description, on the basis of which the embodiments of the invention illustrated in the figures are described and explained in more detail.

In detail:

FIG. 1: shows a schematic section through a pressure transducer according to the invention,

FIG. 2: shows a longitudinal section through a further embodiment of a pressure transducer,

FIG. 3: shows the pressure transducer from FIG. 2 in a rear view onto the base plate with contact faces,

FIG. 4: shows the pressure transducer from FIGS. 2, 3 in a front view,

FIG. 5: shows a schematic section from a cut through a contact holder according to the invention,

FIG. 6: shows a detail of an embodiment for contact pin and sealing membrane,

FIG. 7: shows an exploded view of a contact holder according to the invention, and

FIG. 8: shows the contact holder from FIG. 7 is the assembled state.

### DETAILED DESCRIPTION

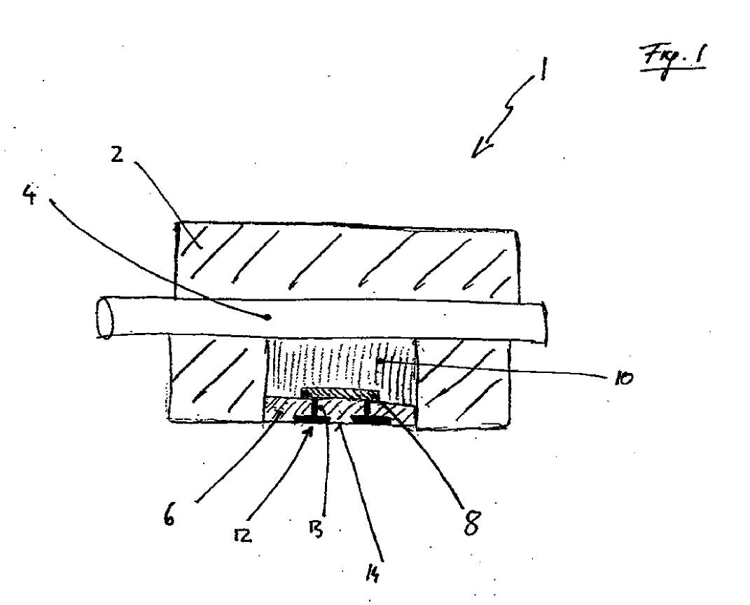

For reasons of clarity, components with mutually corresponding functions are provided with the same reference sign in FIGS. 1-8.

FIG. 1 shows a pressure transducer 1 with a housing section 2. An electrically insulating base plate 6, which is made of a ceramic material in this case, is held in the housing section 2. Here, the base plate 6 is situated in the housing section 2 such that the base plate 6 delimits the housing section 2 to the outside and is freely accessible from the outside. The housing section 2 furthermore includes a fluid chamber 4, which can be connected to the vascular system of a patient by pressure via fluid tubes (not illustrated). A physiological electrolyte solution, e.g. aqueous NaCl solution, is in the fluid chamber 4 as pressure transmitting medium.

In order to determine the pressure prevailing in the fluid chamber 4, provision is made for a pressure measuring element 8, which is applied to the base plate 6 on the side of the latter facing the fluid chamber 4. By way of example, it is feasible that the electronic operating elements of the pressure measuring element 8 are directly defined on the base plate 6 using lithographic methods. Arranged between the pressure measuring element 8 and the fluid chamber 4 there is an elastic pressure pad 10 made of a polysiloxane (silicone), via which the pressure prevailing in the fluid chamber 4 is transmitted onto the pressure measuring element 8. Moreover, the pressure pad 10 seals the electronics of the pressure measuring element 8 from liquid possibly emerging from the fluid chamber 4.

Two metallic, electric contact faces 12 are embedded into the base plate 6 such that the surface of the contact faces 12 and the surface of the base plate 6 distant from the fluid chamber 4 lie in a plane 14, and merge into one another without steps. Thus, the contact faces 12 are accessible from the side of the housing section 2 that is facing away from the pressure measuring element 8 and the fluid chamber 4. The contact faces 12 have conductor pins 13 for electrically contacting the pressure measuring element 8. Here, the contact faces 12 are embedded into the base plate 6 in a liquid-tight fashion and are preferably molded into the ceramic material during the production of the base plate 6 such that the conductor pins 13 pass through the base plate 6 to the pressure measuring element 8. The contact faces 12 and the conductor pins 13 are made of a gold-plated metallic material, e.g. gold-plated copper.

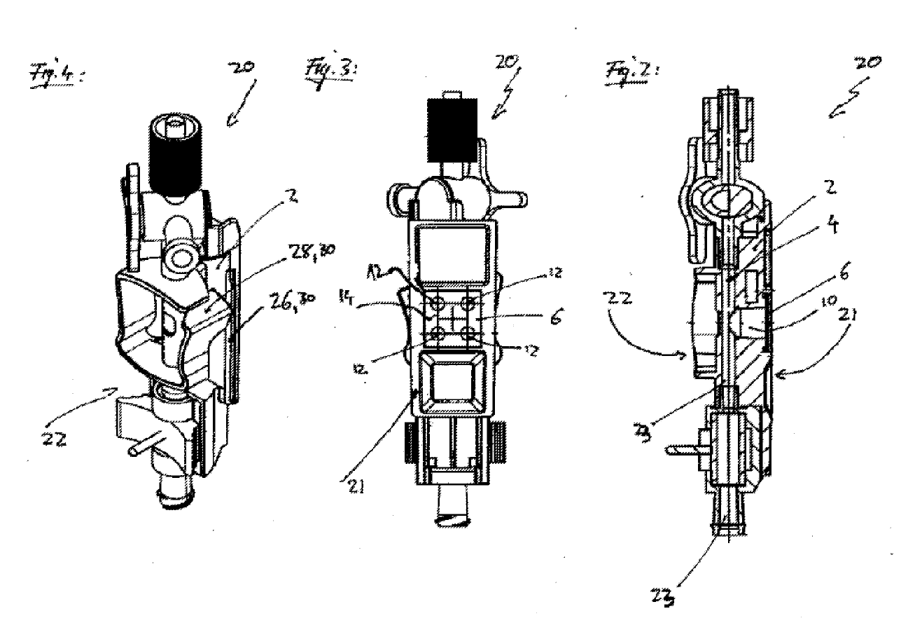

FIG. 2 illustrates a pressure transducer 20, which has a housing 2 with a rear side 21 and a front side 22. A fluid chamber 4 is enclosed in the housing 2 and in this case is provided by a section of a fluid tube 23 for connecting the fluid chamber 4 to the vascular system of a patient. In order to regulate the through-flow of fluid (e.g. aqueous NaCl solution), a fluid valve 24 is arranged in the fluid tube 23.

The pressure transducer 20 moreover comprises a ceramic base plate 6, which is embedded into the housing 2 and delimits the latter on the rear side 21. Arranged between the base plate 6 and the fluid chamber 4 there is an elastic pressure pad 10, made of a polysiloxane (silicone), for transmitting the pressure prevailing in the fluid chamber 4. The section illustrated in FIG. 2 is selected such that the contact faces 12 do not lie in the plane of the section and hence they are not shown.

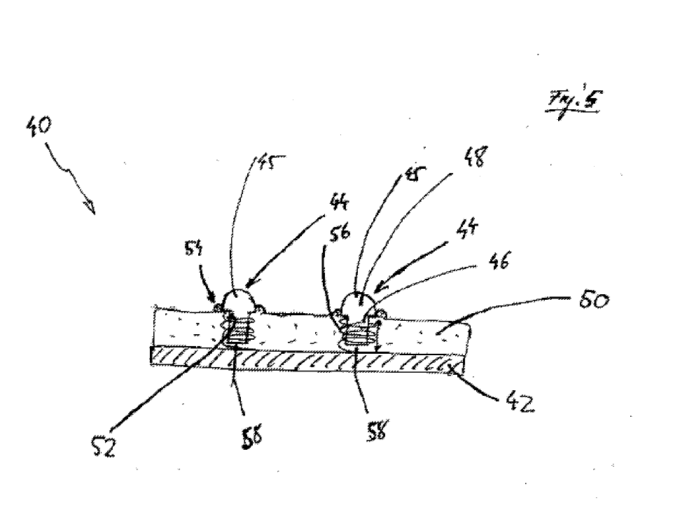

FIG. 3 illustrates a view of the rear side 21 of the housing 2 of the pressure transducer 20 from FIG. 2. It is possible to identify the ceramic base plate 6, into which four electric contact faces 12 made of gold-plated metallic material have been embedded. The surfaces of the contact faces 12 are freely accessible from the rear side 21 and are situated in a step-free plane 14 with the surface of the base plate 6 that closes off the housing 2 to the outside.

FIG. 4 shows the pressure transducer 20 from FIGS. 2, 3 in an oblique view of the front side 22. The housing 2 has a holding section 26, embodied as an undercut edge, by means of which the pressure transducer 20 can be mounted in a mechanically detachable fashion in a contact holder (not illustrated). It is also feasible for the base plate 6 to provide the holding section 26.

The pressure transducer 20 furthermore has a clamping clasp 28, by means of which the pressure transducer 20 can be detachably latched into a contact holder (not illustrated). It follows that the holding section 26 and/or the clamping clasp 28 constitute mechanical holding means 30 for detachably mounting the pressure transducer 20 on a contact holder.

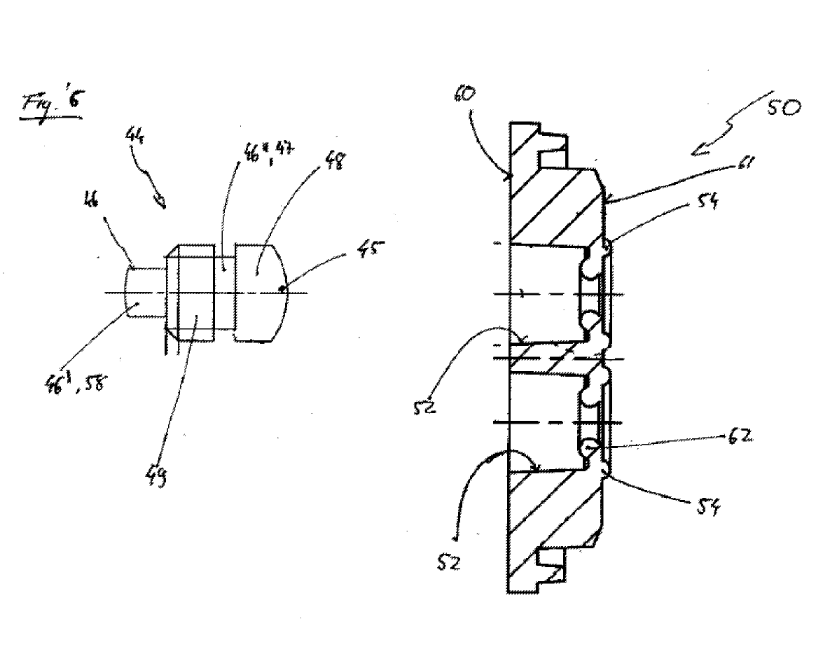

FIG. 5 illustrates part of a schematic section through a contact holder 40 for mounting and electrically contacting a pressure transducer for medical use, as shown in FIGS. 1 to 4. The contact holder 40 has a plate-like support section 42, which is made of hard plastic. Furthermore, provision is made for contact pins 44, which each have one contact tip 45 for electrically contacting contact faces 12 of a pressure transducer 1, 20 (cf. FIGS. 1 to 4).

In order to improve the electrical conductivity, the contact pins 44 are made of a gold-plated, metallic material. The contact pins 44 comprise a cylindrical shaft 46 and a pin head 48 with a greater radial diameter than the shaft 46. Here, the pin head 48 has a mushroom-like design and has the contact tip 45.

Arranged adjoining the plate-like support section 42 there is a sealing membrane 50 made of silicone-like material. This material is elastic, acts in an electrically insulating fashion and repels polar liquids (it is hydrophobic). The sealing membrane 50 has recesses 52, into which respectively one shaft 46 of a contact pin 44 is embedded perpendicularly to the support section 42. Here, the recesses 52 are dimensioned such that the elastic sealing membrane 50 adjoins the respective shaft 46 under tension and thus it seals against liquids. The mushroom-like contact tip 45 of the contact pin 44 adjoins the sealing membrane 50 such that the recess 52 is covered by the pin head 48.

The sealing membrane 50 respectively has one annular sealing bead 54 around a recess 52. The sealing bead 54 adjoins the sealing head 48 and ensures further sealing with respect to liquid.

The contact pins 44 are embedded into the sealing membrane 50 such that the contact tip 45 can be depressed in the direction of the support section 42. Since the pin head 48 adjoins the elastic sealing membrane 50, the contact tip 45 has tensioning preventing a depression. Furthermore, a helical-spring-like spring element 56 is embedded around each shaft 46 into the sealing membrane 50 and it is supported by firstly the support section 42 and secondly the pin head 48 and thus likewise acts on the contact tip 45 in the direction away from the support section 42.

Conductor sections 58 are provided for transmitting electric signals; these conductor sections respectively contact one contact pin 44 electrically and pierce the support section 42 to the side distant from the sealing membrane 50.

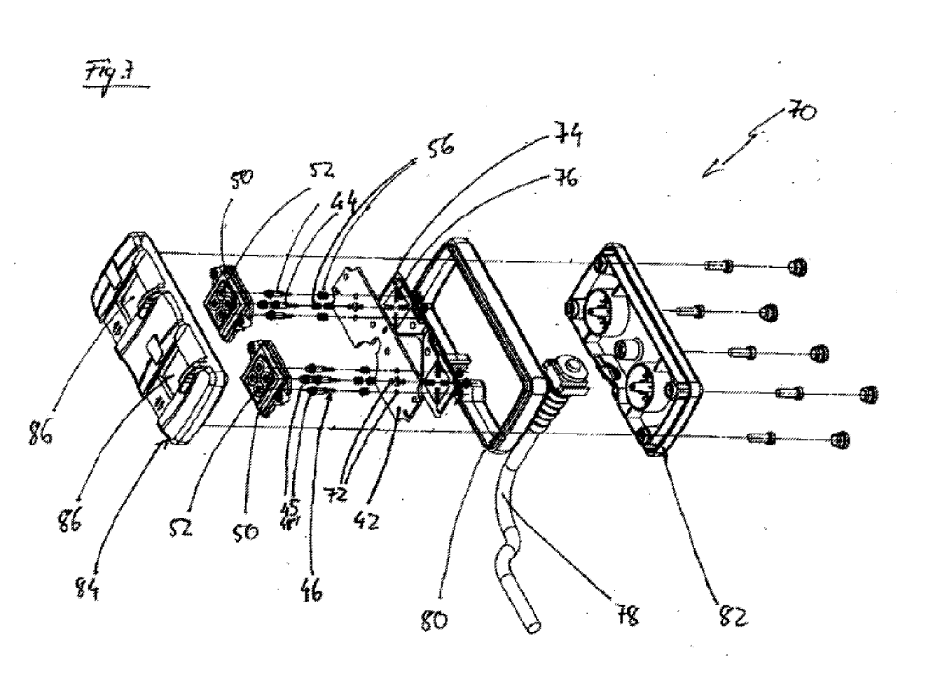

FIG. 6 shows sections through a contact pin 44 and a sealing membrane 50, as can be used for building a contact holder according to the invention.

The contact pin 44 in FIG. 6 has a shaft 46 with shaft sections 46′ and 46″, and also a mushroom-like rounded pin head 48 with a contact tip 45. The shaft section 46′ has a smaller radial diameter than the shaft section 46″, which in turn has a smaller radial diameter than the pin head 48. The transition from shaft section 46′ to section 46″ is formed by a radially enlarged annular collar 49. It follows that the contact pin 44 has an annular groove 47 between the annular collar 49 and the pin head 48; in this case the annular groove 47 is formed by the shaft section 46″.

The sealing membrane 50 has a bearing side 60, along which the sealing membrane 50 is fastened to a support section 42 in order to build a contact holder according to the invention. The sealing membrane 50 has two recesses 52 in, into which respectively one contact pin 44, as shown in FIG. 6, can be embedded. In each recess 52, the recess 52 is constricted radially by an annular projection 62 on the side 61 distant from the bearing side 60. On the side 61, the sealing membrane 50 has a sealing bead 54 around each recess 52.

In order to embed the contact pin 44 into the elastic sealing membrane 50, the contact pin 44 is introduced into the recess 52 in the alignment illustrated in FIG. 6. As a result, the sealing membrane 50 adjoins the annular collar 49 in the region of the recess 52 facing the bearing side 60. The annular projection 62 engages into the annular groove 47 of the contact pin 44, formed between the annular collar 49 and pin head 48. Since the sealing membrane 50 has an elastic design, the contact pin 44 is mounted in the sealing membrane 50 by the annular projection 62 such that it can move in the direction perpendicular to the bearing side 60. The sealing bead 54 adjoins the pin head 48 radially and seals against the ingress of liquid.

In the assembled state, the shaft section 46′ projects beyond the bearing side 60 of the sealing membrane 50 and forms a conductor section 58 for electrically contacting the contact pin 44.

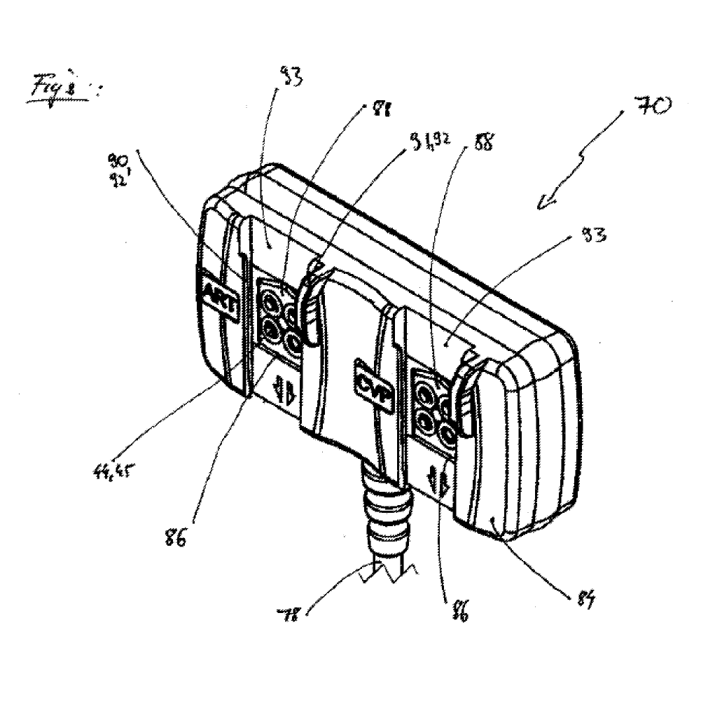

FIG. 7 shows the individual parts in an exploded view for explaining the design of a contact holder 70 according to the invention. The contact holder 70 has a plate-like support section 42 made of hard plastic, into which pin holes 72 have been introduced. Two elastic sealing membranes 50 (cf. FIG. 6) are arranged in an adjoining fashion on the support section 42. Each sealing membrane 50 has recesses 52, into which contact pins 44 are embedded perpendicularly to the plate-like support section 42. The contact pins 44 respectively have mushroom-like pin heads 48 with contact tips 45, which are distant from the support section 42 in the embedded state. Here, the pin heads 48 lie on the surface of the sealing membrane 50. Furthermore, spring elements 55 are arranged around each contact pin 44, which act on the contact tips 45 counter to the support section 42.

Each contact pin 44 moreover has a cylindrical shaft 46, which is embedded into one of the recesses 52 of the sealing membrane 50 in the assembled state and engages through one of the pin holes 72 of the support section 42.

The contact holder 70 furthermore has an electric printed circuit board 74 with electric printed conductors 76. The printed circuit board 74 is arranged adjoining the support section 42 on the side of the latter distant from the sealing membranes 50. Here, each printed conductor 76 accordingly contacts one shaft 46 of a contact pin 44.

A contact cable 78 through which electrical measuring signals can be transmitted is provided for further contacting of the printed circuit board 74.

For enclosing the components, the contact holder 70 has a housing 80, which is completed by a rear plate 82 and a front plate 84.

The front plate 84 has two front recesses 86 for respectively one sealing membrane 50 and hence has four contact pins 44 in each case.

FIG. 8 illustrates the contact holder 70 from FIG. 7 in the assembled state. The front recesses 86 of the front plate 84 expose two contact regions 88 with respectively four contact pins 44. The contact tips 45 of the contact pins 44 are arranged such that they can be depressed under tensioning, as explained above.

The front plate 84 has undercut rails 90 and clamping projections 91. This provides respectively one holding device 92 for respectively one pressure transducer 20 (cf. FIGS. 2 to 4). Furthermore, guide faces 93 are provided in the region of each holding device 92 and these simplify the insertion of a pressure transducer 20.

In order to mount a pressure transducer 20, as illustrated in FIGS. 2 to 4, it is inserted into the contact holder 70 from above such that the base plate 6 glides along the guide faces 93. In the process, the holding section 26 of the pressure transducer 20 (cf. FIG. 4) engages into the undercut rail 90 of the contact holder 70. Once the pressure transducer 20 has been completely inserted, the clamping clasp 28 of the pressure transducer 20 (cf. FIG. 4) latches with the clamping projection 91.

In this position, the base plate 6, with the contact faces 12 of the pressure transducer 20 (cf. FIG. 3), is securely pressed against the contact region 88 of the contact holder 70. Here, respectively one contact face 12 adjoins a corresponding contact tip 45, wherein the contact tip 45, as explained above, can be depressed to a certain extent against tensioning.

Hence, the pressure transducer 20 is detachably mounted and electrically contacted such that the measuring signals of the pressure transducer 20 are provided for transmission by means of the contact cable 78.

1. A pressure transducer for measuring blood pressure for medical use, the pressure transducer comprising:
a fluid chamber which can be connected by pressure to the vascular system of a patient,
a pressure measuring element for measuring the pressure prevailing in the fluid chamber,
a mechanical holding means for detachably mounting the pressure transducer on a contact holder, and
an electrically insulating base plate, the electrically insulating base plate having at least one electric contact that is electrically connected to the pressure measuring element wherein the surfaces of the contact and the base plate substantially lie in a common plane and an electrical contact is established between the contact and an associated counter contact of the contact holder by mounting the pressure transducer in the contact holder.

2. The pressure transducer as claimed in claim 1, wherein the contact is embedded into the base plate in a liquid-tight fashion.

3. The pressure transducer as claimed in claim 1, further comprising an elastic pressure pad arranged for transmitting pressure between the fluid chamber and the pressure measuring element.

4. The pressure transducer as claimed in claim 1, wherein the base plate has electric circuits of the pressure measuring element.

5. The pressure transducer as claimed in claim 1, wherein the fluid chamber and the pressure measuring element are arranged together on one side of the base plate and the contact face is accessible from the side of the base plate distant from the fluid chamber.

6. The pressure transducer as claimed in claim 1, further comprising a fluid tube for connecting the pressure transducer to the patient and in that the fluid chamber is provided by a section of the fluid tube.

7. The pressure transducer as claimed in claim 1, wherein the base plate includes a holding section, which provides the holding means for the pressure transducer.

8. A contact holder for mounting and electrically contacting a pressure transducer for medical use, the contact holder comprising:
a plate-like support section,
which provides at least one contact pin, which has a contact tip as an electrical counter contact for a contact face of the pressure transducer,
wherein the contact pin is arranged substantially perpendicular to the support section such that the contact tip is distant from the support section and contacts the associated contact face if the pressure transducer is mounted,
a sealing membrane made of an elastic, hydrophobic and electrically insulating material, the sealing membrane arranged adjoining the support section and having a recess for respectively one contact pin such that the contact pin is embedded into the sealing membrane in a liquid-tight fashion and the contact tip remains uncovered by the sealing membrane.

9. The contact holder as claimed in claim 8, wherein, around the recess, the sealing membrane has an annular sealing bead that adjoins the contact pin in a sealing fashion.

10. The contact holder as claimed in claim 8, wherein the contact pin comprises a cylindrical shaft and a pin head with a greater radial diameter than the shaft, wherein the pin head has the contact tip and adjoins the sealing membrane and wherein the sealing membrane encloses the shaft in a liquid-tight fashion.

11. The contact holder as claimed in claim 8, wherein the contact tip has a mushroom-like design.

12. The contact holder as claimed in claim 8, wherein the contact pin is provided on the support section such that the contact tip can be depressed in the direction of the support section against tensioning applied by a spring element.

13. The contact holder as claimed in claim 8, further comprising a holding device configured such that a pressure transducer can be inserted parallel to the support section for the purpose of being detachably mounted on the contact holder.

14. The contact holder as claimed in claim 8, further comprising an electric printed circuit board, which is arranged adjoining the support section or parallel to the support section on the side distant from the sealing membrane such that the contact pin is electrically contacted.

15. The contact holder as claimed in claim 8, wherein the contact pin has an annular groove and, in the region of the recess, the sealing membrane has an annular projection, which engages into the annular groove.

16. A pressure transducer arrangement comprising a pressure transducer as claimed in claim 1 and a contact holder as claimed claim 8.
